(19) United States
(12) Patent Application Publication
(10) Pub. No.: US 2019/0160301 A1
(43) Pub. Date: May 30, 2019

(54) LASER BEAM DEVICE AND LASER BEAM HAND PIECE

(71) Applicant: LUTRONIC CORPORATION, Goyang (KR)

(72) Inventor: Hee Chul LEE, Goyang (KR)

(21) Appl. No.: 16/097,559

(22) PCT Filed: Apr. 28, 2017
(86) PCT No.: PCT/KR2017/004567

§ 371 Date: Oct. 29, 2018

(30) Foreign Application Priority Data
Apr. 29, 2016 (KR) 10-2016-0053042

(51) Int. Cl. A61N 5/06

(52) U.S. Cl. CPC A61N 5/0616; A61N 2005/067; A61N 2005/0644

(57) ABSTRACT

The present invention relates to a laser beam device outputting a multi-spot laser beam, and a laser bean hand piece. The laser beam device, according to the present invention, comprises: a light source unit which generates and emits a single-spot laser beam having a first wavelength; a laser beam oscillation unit which outputs the first wavelength single-spot laser beam emitted from the light source unit as any one of a first wavelength multi-spot laser beam and a single-spot laser beam having a second wavelength, and then outputs any one of the outputted first wavelength multi-spot laser beam and second wavelength single-spot laser beam as a second wavelength multi-spot laser beam, and an amplification unit which amplifies the second wavelength multi-spot laser beam so that the output energy of the second wavelength multi-spot laser beam outputted from the laser beam oscillation unit is relatively high output energy.

### TECHNICAL FIELD

The present invention relates to a laser beam device and a laser beam handle piece and, more particularly, to a laser beam device and a laser beam handle piece for emitting a laser beam.

### BACKGROUND ART

A laser beam device is a device for outputting a laser beam having three characteristics including monochromatic, coherence, and collimation unlike general nature light and light emitted from a lamp. The laser beam is outputted with an energy having a different wavelength or a different pulse width according to change of an oscillation condition of the laser beam.

The laser beam outputted from the laser beam device has excellent characteristics of monochromatic, coherence, and collimation to have been widely used in various industrial fields. For example, the laser beam device has been used in various industrial fields such as a metal industry, a construction industry, a shipbuilding industry and a medical industry. In particular, usability of the laser beam device has been increased in a medical industrial field according to increase of cure efficiency due to emission of a laser beam.

Meanwhile, a laser beam device used in the medical industrial field outputs a laser beam having various wavelengths, pulse widths or output energies according to various usages such as cure purpose, cure parts, or beauty purpose.

A representative laser beam device in a medical industrial field is widely used for skin cure or skin beauty. A laser beam outputted from the laser beam device used for the purpose of skin cure or skin beauty generally uses a single wavelength laser beam and a single-spot laser beam.

Accordingly, there is a need to develop a laser beam device capable of a wavelength, a spot, or an output energy of a laser beam outputted in order to increase usability for cure or beauty.

### DISCLOSURE

### Technical Problem

An embodiment of the present invention provides a laser beam device and a laser beam hand piece having an improved structure capable of changing a wavelength, a spot, and output energy of an input laser beam.

### Technical Solution

In accordance with an aspect of the present invention, there is provided a laser beam device including: a light source unit which generates and emits a single-spot laser beam having a first wavelength; a laser beam oscillation unit which outputs the first wavelength single-spot laser beam emitted from the light source unit as any one of a first wavelength multi-spot laser beam and a single-spot laser beam having a second wavelength, and then outputs any one of the outputted first wavelength multi-spot laser beam and second wavelength single-spot laser beam as a second wavelength multi-spot laser beam, and an amplification unit which amplifies the second wavelength multi-spot laser beam so that the output energy of the second wavelength multi-spot laser beam outputted from the laser beam oscillation unit is relatively high output energy.

The laser beam oscillation unit may include: a multi-spot forming part configured to output the first wavelength single-spot laser beam input as the first wavelength multi-spot laser beam; and an optical part disposed oppositely to the light source unit while interposing the multi-spot forming part therebetween to output the first wavelength multi-spot laser beam as the second multi-spot laser beam.

The laser beam oscillation unit may include: an optical part configured to output the first wavelength single-spot laser beam input and pumped as the second wavelength single-spot laser beam; and a multi-spot forming part disposed oppositely to the light source unit while interposing the optical part therebetween to output the second wavelength single-spot laser beam input as the second multi-spot laser beam.

The amplification unit may include: an amplification optical part configured to absorb the second wavelength multi-spot laser beam input from the laser beam oscillation unit; and an amplification light source part disposed at a transverse direction of an input direction of the second wavelength multi-spot laser beam input from the amplification optical part to provide a pumping light to the amplification optical part in order to increase output energy of the second wavelength multi-spot laser beam absorbed by the amplification optical part.

The laser beam device may further include a spot adjustment unit disposed oppositely to the laser beam oscillation unit while interposing the amplification unit therebetween to adjust any one of a spot size and a spot interval with respect to the second wavelength multi-spot laser beam outputted from the amplification unit.

The pumping light provided to the amplification optical part from the amplification light source part may have a first wavelength.

The multi-spot forming part may include any one of a micro-lens array (MLA) and a diffractive optical element (DOE).

The laser beam device may further include an optical unit disposed oppositely to the laser beam oscillation unit while interposing the amplification unit therebetween to output the second wavelength multi-spot laser beam outputted from the amplification unit as a third wavelength multi-spot laser beam.

In accordance with another aspect of the present invention, there is provided a laser beam hand piece including: a light source unit which generates and emits a single-spot laser beam having a first wavelength; a laser beam oscillation unit including a multi-spot forming part configured to output the first wavelength single-spot laser beam emitted from the light source unit as a multi-spot laser beam, and an optical part configured to output a first wavelength multi-spot laser beam as a second wavelength multi-spot laser beam; and an amplification unit including an amplification optical part configured to absorb the second wavelength multi-spot laser beam input from the laser beam oscillation unit, and an amplification optical part configured to provide a pumping light to the amplification optical part in order to increase output energy of the second wavelength multi-spot laser beam by the amplification optical part.

The laser beam hand piece may further include a spot adjustment unit disposed oppositely to the laser beam oscillation unit while interposing the amplification unit therebetween to adjust any one of a spot size and a spot interval with respect to the second wavelength multi-spot laser beam outputted from the amplification unit.

The amplification light source part may be disposed at a transverse direction of the amplification optical part and may provide a pumping light to a transverse direction of a course of the second wavelength multi-spot laser beam output input to the amplification optical part and outputted from the amplification optical part.

The multi-spot forming part may include any one of a micro-lens array (MLA) and a diffractive optical element (DOE).

The laser beam hand piece may further include an optical unit disposed oppositely to the laser beam oscillation unit while interposing the amplification unit therebetween to output the second wavelength multi-spot laser beam outputted from the amplification unit as a third wavelength multi-spot laser beam.

The pumping light provided to the amplification optical part from the amplification light source part may have a first wavelength.

Each of the optical part and the amplification optical part may include Nd:YAG.

The details of other embodiments are contained in the detailed description and accompanying drawings.

### Advantageous Effects

The laser beam device and the laser beam hand piece according to the present invention have advantages as follows.

First, a single-spot laser beam emitted from a light source unit is wavelength-converted into a different wavelength multi-spot laser beam and output energy may be increased so that various applications of the product are possible.

Second, diversity of the product may be ensured by adjusting the spot size or the spot interval of the multi-spot laser beam.

### DESCRIPTION OF DRAWINGS

FIG. 1 is a sectional view illustrating a laser beam device according to a first embodiment of the present invention;

FIG. 2 is a schematic view illustrating a configuration of the laser beam device shown in FIG. 1;

FIG. 3 is a schematic view illustrating another configuration of the laser beam device shown in FIG. 1;

FIG. 4A and FIG. 4B are first pattern adjustment diagrams of a multi-spot laser beam outputted from the laser beam device shown in FIG. 1, respectively;

FIG. 5A and FIG. 5B are second pattern adjustment diagrams of a multi-spot laser beam outputted from the laser beam device shown in FIG. 1, respectively;

FIG. 6 is a sectional view illustrating a laser beam device according to a second embodiment of the present invention; and

FIG. 7 is a schematic view illustrating a configuration of the laser beam device shown in FIG. 6.

### MODE FOR INVENTION

Hereinafter, a laser beam device and a laser beam handle piece according to the exemplary embodiment of the present invention will be described with reference to the accompanying drawings.

Before the description, a laser beam handle piece is shown as a laser beam device according to first and second embodiments of the present invention. However, various devices may be used in addition to the laser beam hand piece.

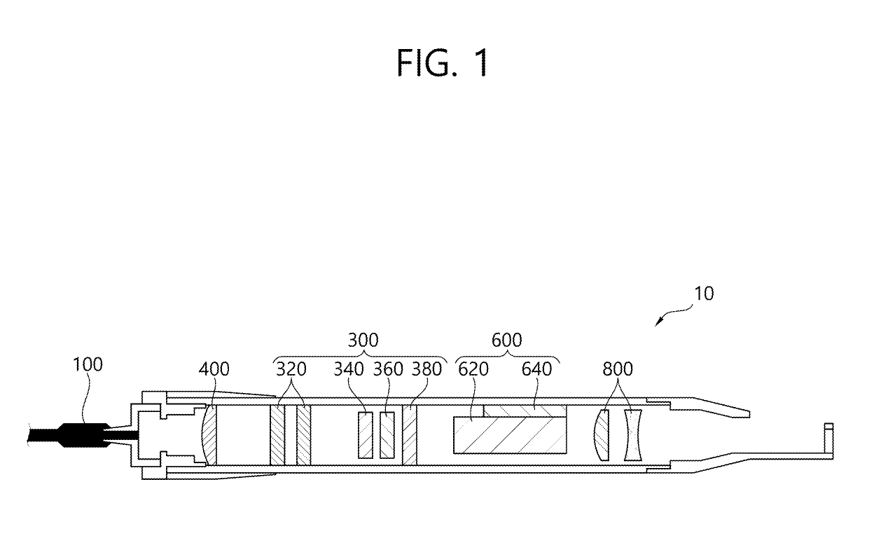

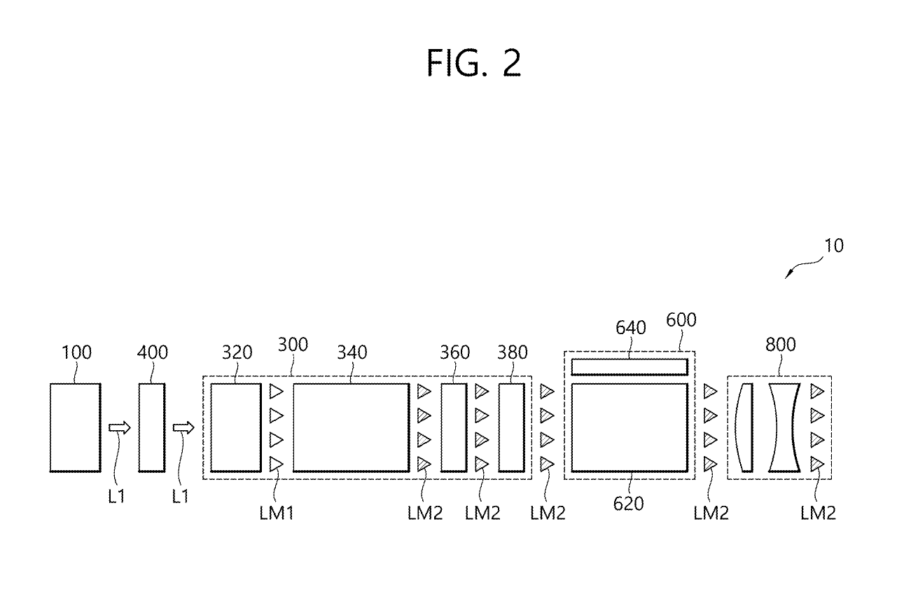

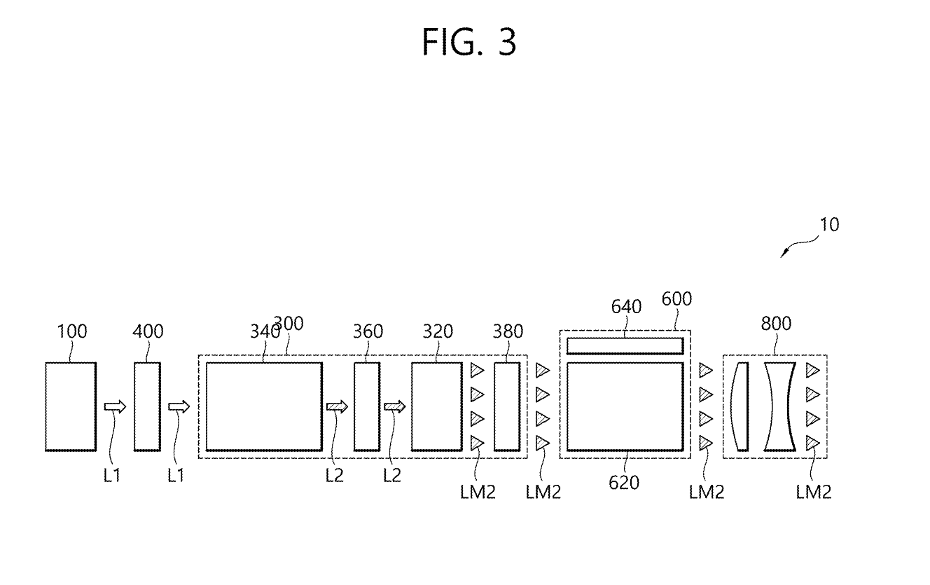

FIG. 1 is a sectional view illustrating a laser beam device according to a first embodiment of the present invention, FIG. 2 is a schematic view illustrating a configuration of the laser beam device shown in FIG. 1, and FIG. 3 is a schematic view illustrating another configuration of the laser beam device shown in FIG. 1.

As shown in FIG. 1 to FIG. 3, the laser beam device 10 according to a first embodiment of the present invention includes a light source unit 100, a laser beam oscillation unit 300, and an amplification unit 600. The laser beam device 10 according to a first embodiment of the present invention further includes a collimation unit 400 and a spot adjustment unit 800.

The light source unit 100 generates and generates a laser beam to the laser beam oscillation unit 300. The light source unit 100 includes an optical fiber as an example of the present invention, and generates and emits a first wavelength single-spot laser beam L1 of 808 nm to the laser beam oscillation unit 300. The light source unit 100 may use various configurations such as a laser diode (LD) array capable of generating and emitting a first wavelength single-spot laser beam L1.

As shown in FIG. 2, the laser beam oscillation unit 300 outputs the first wavelength single-spot laser beam L1 emitted from the light source unit 100 as a multi-spot laser beam, and then outputs a first wavelength multi-spot laser beam LM1 as a second wavelength multi-spot laser beam LM2. As shown in FIG. 3, the laser beam oscillation unit 300 outputs the first wavelength single-spot laser beam L1 emitted from the light source unit 100 as a second wavelength single-spot laser beam L2, and then outputs a second wavelength single-spot laser beam L2 as a second wavelength multi-spot laser beam LM2. That is, the laser beam oscillation unit 300 outputs a single-spot laser beam as a multi-spot laser beam according to locations of a multi-spot forming part 320 and an optical unit 340 of the laser beam oscillation unit 300 and then converts the wavelength thereof or converts the wavelength and then outputs the single-spot laser beam as a multi-spot laser beam.

The laser beam oscillation unit 300 includes a multi-spot forming part 320 and an optical unit 340. The laser beam oscillation unit 300 further includes a saturated absorber 360 and an output part 380. As shown in FIG. 2, the multi-spot forming part 320, the optical part 340, the saturated absorber 360 and the output part 380 are disposed in the order of the multi-spot forming part 320, the optical part 340, the saturated absorber 360 and the output part 380 in the laser beam oscillation unit 300.

First, the multi-spot forming part 320, the optical part 340, the saturated absorber 360 and the output part 380 shown in FIG. 2 will be described as follows.

The multi-spot forming part 320 outputs a first wavelength single-spot laser beam L1 input from the light source unit 100 as a first wavelength multi-spot laser beam LM1. The multi-spot forming part 320 includes any one of a micro-lens array (MLA) and a diffractive optical element (DOE).

The optical part 340 wavelength-converts a first wavelength multi-spot laser beam LM1 formed in the multi-spot forming part 320 into a second wavelength multi-spot laser beam LM2 to output the second wavelength multi-spot laser beam LM2.

The optical unit 340 includes Nd:YAG. The optical unit 340 absorbs a first wavelength multi-spot laser beam LM1 of 808 nm according to coating of an input surface to output a second wavelength multi-spot laser beam LM2 of 1064 nm.

The saturated absorber 360 saturation-absorbs and outputs the second wavelength multi-spot laser beam LM2 outputted from the optical part 340. As an example of the present invention, the saturated absorber 360 uses Cr:YAG.

The output part 380 is configured by an output coupler (OC) mirror and amplifies and oscillates the second wavelength multi-spot laser beam LM2 generated from the optical part 340 and the saturated absorber 360. The output part 380 is configured by a general flat mirror and oscillates the amplified second wavelength multi-spot laser beam LM2.

As shown in FIG. 3, the optical part 340, the saturated absorber 360, the multi-spot forming part 320, and the output part 380 are disposed in the order of the optical part 340, the saturated absorber 360, the multi-spot forming part 320, and the output part 380 in the laser beam oscillation unit 300.

The optical part 340 absorbs the first wavelength single-spot laser beam L1 provided from the light source unit 100 to output the second wavelength single-spot laser beam L2. Further, the multi-spot forming part 320 outputs the second wavelength single-spot laser beam L2 as a second wavelength multi-spot laser beam LM2.

Here, the laser beam oscillation unit 300 may oscillate a laser beam having a pico-second pulse width or a nano-second pulse width according to a disposal length of constituent elements. Alternatively, the laser beam oscillation unit 300 may oscillate a laser beam having a nano-second pulse width by adjusting optical properties of the saturated absorber 360 or a reflectivity of the output part 380. For example, if a length of the laser beam oscillation unit 300 is 1 cm, the laser beam oscillation unit 300 may oscillate a laser beam having a pico-second pulse width. If increasing the length of the laser beam oscillation unit 300 as several cm, the laser beam oscillation unit 300 may oscillate a laser beam having a nano-second pulse width.

The collimation unit 400 is disposed between the light source unit 100 and the laser beam oscillation unit 300 to maintain collimation, that is, balance of the first wavelength single-spot laser beam L1 emitted from the light source unit 100.

Next, the amplification unit 600 amplifies the second wavelength multi-spot laser beam LM2 so that the output energy of the second wavelength multi-spot laser beam LM2 outputted from the laser beam oscillation unit 300 is relatively high output energy. Since the output energy of the second wavelength multi-spot laser beam LM2 oscillated from the laser beam oscillation unit 300 is very low, the second wavelength multi-spot laser beam LM2 cannot be used as a medical laser beam so that the amplification unit 600 is used. For example, if it is assumed that a second wavelength multi-spot laser beam LM2 having 100 multi-spots is outputted from the laser beam oscillation unit 300, each output energy of the second multi-spot laser beam LM2 has a very low output energy of about 100 uJ so that the second multi-spot laser beam LM2 cannot be used for skin cure. Accordingly, the amplification unit 600 amplifies each second wavelength multi-spot laser beam LM2 having output energy of 100 uJ as an output energy of several mJ to several tens mJ.

As an embodiment of the present invention, the amplification unit 600 includes an amplification optical part 620 and an amplification light source part 640. The amplification optical part 620 absorbs the second wavelength multi-spot laser beam LM2 oscillated and input from the laser beam oscillation unit 300. Further, the amplification light source part 640 is disposed at a transverse direction of an input direction of the second wavelength multi-spot laser beam LM2 input from the amplification optical part 620 and provides a pumping light to the amplification optical part 620 in order to increase an output energy of the second wavelength multi-spot laser beam LM2 absorbed from the amplification optical part 620. The amplification optical part 620 is configured by a laser diode (LD) bar and provides a pumping light to the amplification optical part 620 in a transverse direction of a course of the second wavelength multi-spot laser beam LM2.

Here, the amplification optical part 620 includes Nd:YAG like the optical part 340 of the laser beam oscillation unit 300. The amplification light source part 640 provides a first wavelength light having the same wavelength as that of the first wavelength single-spot laser beam L1 emitted from the light source unit 100 to the amplification optical part 620 as the pumping light. That is, the amplification light source part 640 provides a pumping light of 808 nm to the amplification optical part 620 as an example. An amplification rate of output energy of the second wavelength multi-spot laser beam LM2 amplified from the amplification unit 600 is determined according to progress of the second wavelength multi-spot laser beam LM2.

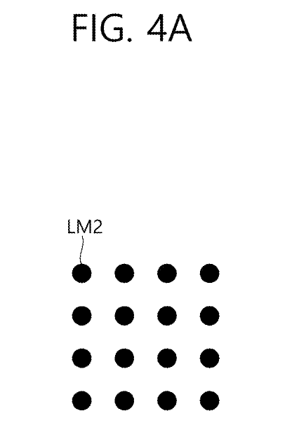

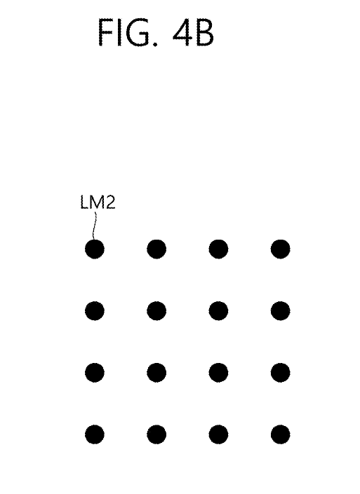

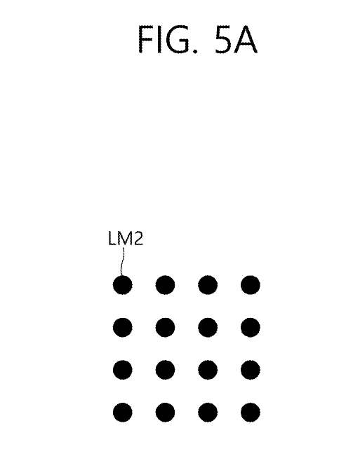

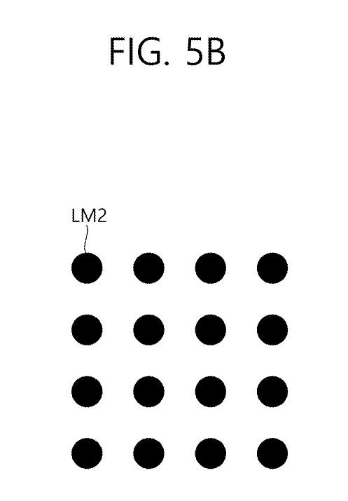

FIG. 4 illustrates first pattern adjustment diagrams of a multi-spot laser beam outputted from the laser beam device shown in FIG. 1, and FIG. 5 illustrates second pattern adjustment diagrams of a multi-spot laser beam outputted from the laser beam device shown in FIG. 1.

The spot adjustment unit 800 is disposed oppositely to the laser beam oscillation unit 300 while interposing the amplification unit 600 therebetween. The spot adjustment unit 800 adjusts any one of a spot size and a spot interval with respect to the second wavelength multi-spot laser beam LM2 outputted from the amplification unit 600. As shown in FIG. 4A and FIG. 4B, the spot adjustment unit 800 adjusts an interval of the second wavelength multi-spot laser beam LM2. As shown in FIG. 5A and FIG. 5B, the spot adjustment unit 800 adjusts a spot size of the second wavelength multi-spot laser beam LM2. The spot adjustment unit 800 may output the second wavelength multi-spot laser beam LM2 from the amplification unit 600 without adjusting the spot interval or the spot size. The spot adjustment unit 800 is configured by a convex lens or a concave lens, and adjusts the spot interval or the spot size in the same principle as that of an aperture of a camera.

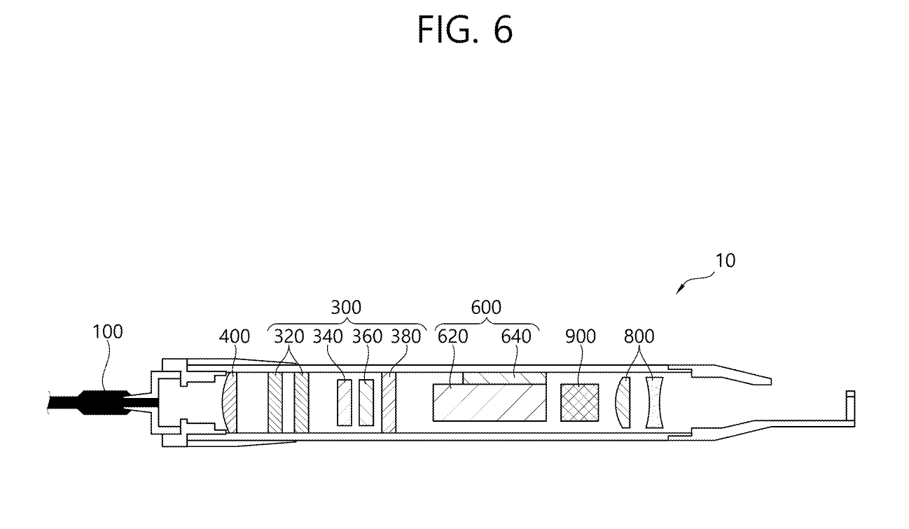

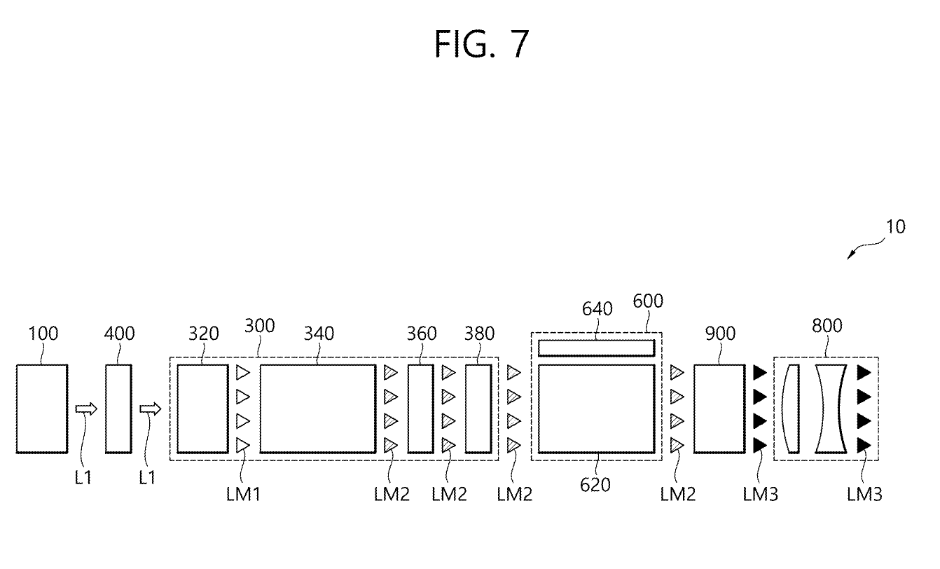

FIG. 6 is a sectional view illustrating a laser beam device according to a second embodiment of the present invention and FIG. 7 is a schematic view illustrating a configuration of the laser beam device shown in FIG. 6.

The laser beam device 100 according to the second embodiment of the present invention includes a light source unit 100, a laser beam oscillation unit 300, a collimation unit 400, an amplification unit 600, and a spot adjustment unit 800 like the first embodiment of the present invention and further includes an optical unit 900. Moreover, the arrangement of lower constituent elements of the laser beam oscillation unit 300 of the laser beam device 10 according to a second embodiment of the present invention may be changed and applied as in the first embodiment. Hereinafter, since constituent elements different from the optical unit 900 were described in the laser beam device 100 according to the first embodiment of the present invention, a detailed description thereof will be omitted.

As shown FIG. 6 and FIG. 7, the optical unit 900 is disposed oppositely to the laser beam oscillation unit 300 while interposing the amplification unit 600 therebetween. The optical unit 900 wavelength-converts the second wavelength multi-spot laser beam LM2 outputted from the amplification unit 600 into a third wavelength multi-spot laser beam LM3. The optical unit 900 uses non-linear crystal such as KTP as an example of the present invention. As an example, the optical unit 900 wavelength-converts the second wavelength multi-spot laser beam LM2 into a third wavelength multi-spot laser beam LM3 of 532 nm to output the third wavelength multi-spot laser beam LM3.

As shown in FIG. 4 and FIG. 5, the spot interval or the spot size of the third wavelength multi-spot laser beam LM3 outputted from the optical unit 900 may be adjusted by the spot adjustment unit 800.

### INDUSTRIAL APPLICABILITY

Accordingly, since the single-spot laser beam emitted from the light source unit may be converted into a different wavelength multi-spot laser beam and output energy may be increased, various applications of the product are possible.

Further, the diversity of the product may be ensured by adjusting the spot size or the spot interval of the multi-spot laser beam.

Although embodiments have been described with reference to a number of illustrative embodiments thereof, it should be understood that numerous other modifications and embodiments can be devised by those skilled in the art that will fall within the spirit and scope of the principles of this disclosure. More particularly, various variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

1. A laser beam device comprising:
a light source unit which generates and emits a single-spot laser beam having a first wavelength;
a laser beam oscillation unit which outputs the first wavelength single-spot laser beam emitted from the light source unit as any one of a first wavelength multi-spot laser beam and a single-spot laser beam having a second wavelength, and then outputs any one of the outputted first wavelength multi-spot laser beam and second wavelength single-spot laser beam as a second wavelength multi-spot laser beam, and
an amplification unit which amplifies the second wavelength multi-spot laser beam so that the output energy of the second wavelength multi-spot laser beam outputted from the laser beam oscillation unit is relatively high output energy.

2. The laser beam device of claim 1, wherein the laser beam oscillation unit comprises:
a multi-spot forming part configured to output the first wavelength single-spot laser beam input as the first wavelength multi-spot laser beam; and
an optical part disposed oppositely to the light source unit while interposing the multi-spot forming part therebetween to output the first wavelength multi-spot laser beam as the second multi-spot laser beam.

3. The laser beam device of claim 1, wherein the laser beam oscillation unit comprises:
an optical part configured to output the first wavelength single-spot laser beam input and pumped as the second wavelength single-spot laser beam; and
a multi-spot forming part disposed oppositely to the light source unit while interposing the optical part therebetween to output the second wavelength single-spot laser beam input as the second multi-spot laser beam.

4. The laser beam device of claim 1, wherein the amplification unit comprises:
an amplification optical part configured to absorb the second wavelength multi-spot laser beam input from the laser beam oscillation unit; and
an amplification light source part disposed at a transverse direction of an input direction of the second wavelength multi-spot laser beam input from the amplification optical part to provide a pumping light to the amplification optical part in order to increase output energy of the second wavelength multi-spot laser beam absorbed by the amplification optical part.

5. The laser beam device of claim 1, further comprising a spot adjustment unit disposed oppositely to the laser beam oscillation unit while interposing the amplification unit therebetween to adjust any one of a spot size and a spot interval with respect to the second wavelength multi-spot laser beam outputted from the amplification unit.

6. The laser beam device of claim 4, wherein the pumping light provided to the amplification optical part from the amplification light source part has a first wavelength.

7. The laser beam device of claim 2, wherein the multi-spot forming part comprises any one of a micro-lens array (MLA) and a diffractive optical element (DOE).

8. The laser beam device of claim 1, further comprising an optical unit disposed oppositely to the laser beam oscillation unit while interposing the amplification unit therebetween to output the second wavelength multi-spot laser beam outputted from the amplification unit as a third wavelength multi-spot laser beam.

9. A laser beam hand piece comprising:
a light source unit which generates and emits a single-spot laser beam having a first wavelength;
a laser beam oscillation unit including a multi-spot forming part configured to output the first wavelength single-spot laser beam emitted from the light source unit as a multi-spot laser beam, and an optical part configured to output a first wavelength multi-spot laser beam as a second wavelength multi-spot laser beam; and
an amplification unit including an amplification optical part configured to absorb the second wavelength multi-spot laser beam input from the laser beam oscillation unit, and an amplification optical part configured to provide a pumping light to the amplification optical part in order to increase output energy of the second wavelength multi-spot laser beam by the amplification optical part.

10. The laser beam hand piece of claim 9, further comprising a spot adjustment unit disposed oppositely to the laser beam oscillation unit while interposing the amplification unit therebetween to adjust any one of a spot size and a spot interval with respect to the second wavelength multi-spot laser beam outputted from the amplification unit.

11. The laser beam hand piece of claim 9, wherein the amplification light source part is disposed at a transverse direction of the amplification optical part and provides a pumping light to a transverse direction of a course of the second wavelength multi-spot laser beam output input to the amplification optical part and outputted from the amplification optical part.

12. The laser beam hand piece of claim 9, wherein the multi-spot forming part comprises any one of a micro-lens array (MLA) and a diffractive optical element (DOE).

13. The laser beam hand piece of claim 9, further comprising an optical unit disposed oppositely to the laser beam oscillation unit while interposing the amplification unit therebetween to output the second wavelength multi-spot laser beam outputted from the amplification unit as a third wavelength multi-spot laser beam

14. The laser beam hand piece of claim 9, wherein the pumping light provided to the amplification optical part from the amplification light source part has a first wavelength.

15. The laser beam hand piece of claim 9, each of the optical part and the amplification optical part comprises Nd:YAG.
